(19) United States
(12) Patent Application Publication
(10) Pub. No.: US 2012/0090626 A1
(43) Pub. Date: Apr. 19, 2012

(54) Earplug with embedded metal member

(76) Inventor: Lin-Kuo Chen, Taipei (TW)

(21) Appl. No.: 12/923,974

(22) Filed: Oct. 19, 2010

(51) Int. Cl. A61F 11/00

(52) U.S. Cl. 128/864

(57) ABSTRACT

An earplug includes a plug portion, a handle portion, and a metal-made hollow round tube. The plug portion includes a plurality of domed flanges and rearward extends to a front end of the handle portion. The handle portion extends rearward by a predetermined length to internally define an axially extended hollow bore with an end cap externally formed around a rear end of the handle portion. A radially inward extended flange is formed in around the hollow bore near the rear end of the handle portion. The metal-made hollow round tube has two open ends and is fixedly embedded in the hollow bore of the handle portion near the end cap in the process of manufacturing the earplug. The flange in the hollow bore prevents the metal round tube from moving out of the hollow bore.

### FIELD OF THE INVENTION

The present invention relates to an earplug with embedded metal member, and more particularly to an earplug having a metal round tube disposed in a handle portion of the earplug, so that the earplug can provide an intended function, such as noise reduction, and is detectable to enable easy location of it in the event it is carelessly dropped.

### BACKGROUND OF THE INVENTION

To avoid damaged ear drums, people who work in a highly noisy environment are usually requested to wear earplugs during working. Earplugs for using in such noisy working environment are referred to as industrial earplugs, and are frequently used in, for example, a food processing plant. The food processing plant is a very special working environment, in which a plurality of large-scale tanks or pools for holding liquids is provided. It is very often the workers' earplugs are carelessly dropped into these tanks, pools or even food and meat in the course of food processing; and it would be very difficult to locate the dropped earplugs in such working environment. To avoid the above undesirable situation from occurring, there are codes established for the earplugs to be worn by workers in the food processing plant and other similar industrial working environments. More specifically, the earplugs for use by workers in the food processing plants and similar working environments must include a detectable metal member made of a corrosion-resistant metal material.

FIG. 1 is an assembled perspective view of a conventional earplug currently being frequently used in food processing and other similar plants. As shown, the conventional earplug includes a plug portion 1 and a handle portion 11. The plug portion 1 is molded to include a plurality of axially sequentially arranged domed flanges 12, 13, 14 and 15. The handle portion 11 is located behind the domed flanges 12-15. A metal member 16 having an opening is fitted around the handle portion behind the last flange 15. Since the metal member 16 is detectable with a suitable metal detector, any carelessly dropped earplug can be immediately located using a metal detector.

While the conventional earplug includes a detectable metal member 16 initially stably fitted around the handle portion 16 behind the domed flange 15, the metal member 16 tends to become loosened and even separated from the handle portion 11 after the earplug has been used, touched or impacted many times.

It is therefore tried by the inventor to develop an improved earplug to overcome the drawbacks in the conventional earplug.

### SUMMARY OF THE INVENTION

A primary object of the present invention is to provide an earplug, which includes a metal-made hollow round tube embedded in a hollow bore of a handle portion of the earplug without the risk of moving out of the handle portion, so that the earplug is detectable using a suitable metal detector when the earplug is carelessly dropped.

To achieve the above and other objects, the earplug according to a preferred embodiment of the present invention includes a plug portion, a handle portion, and a metal-made hollow round tube. The plug portion includes a plurality of axially sequentially arranged domed flanges and rearward extends to a front end of the handle portion. The handle portion extends rearward by a predetermined length to internally define an axially extended hollow bore with a radially outward extended end cap externally formed around a rear end of the handle portion. A radially inward extended flange is formed in around the hollow bore near the rear end of the handle portion. The metal-made hollow round tube has two open ends and is fixedly embedded in the hollow bore of the handle portion near the end cap in the process of manufacturing the earplug. With the flange provided in the hollow bore, the metal round tube is prevented by the flange from moving out of the hollow bore. Therefore, the earplug can provide its intended function and is detectable with a suitable metal detector in the event the earplug is carelessly dropped or lost.

### BRIEF DESCRIPTION OF THE DRAWINGS

The structure and the technical means adopted by the present invention to achieve the above and other objects can be best understood by referring to the following detailed description of the preferred embodiments and the accompanying drawings, wherein

FIG. 1 is an assembled perspective view of a conventional earplug;

FIG. 2 is an assembled perspective view of an earplug according to a preferred embodiment of the present invention;

FIG. 3 is an assembled longitudinal sectional view of the earplug of FIG. 2; and

FIG. 4 shows two earplugs of the present invention are connectable to an extended cord for use.

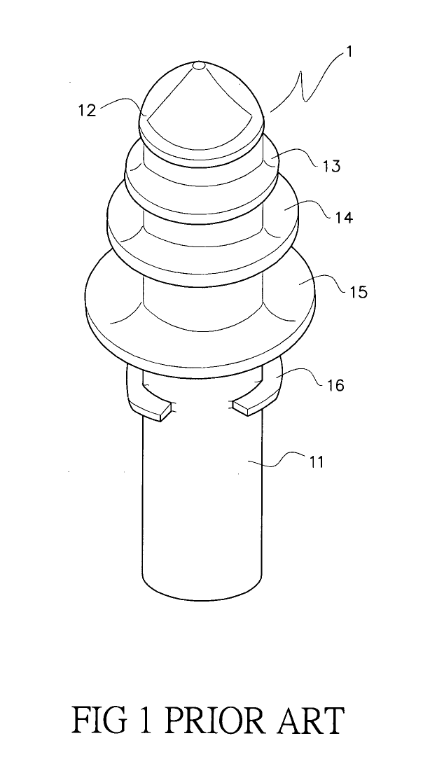

### DETAILED DESCRIPTION OF THE PREFERRED EMBODIMENTS

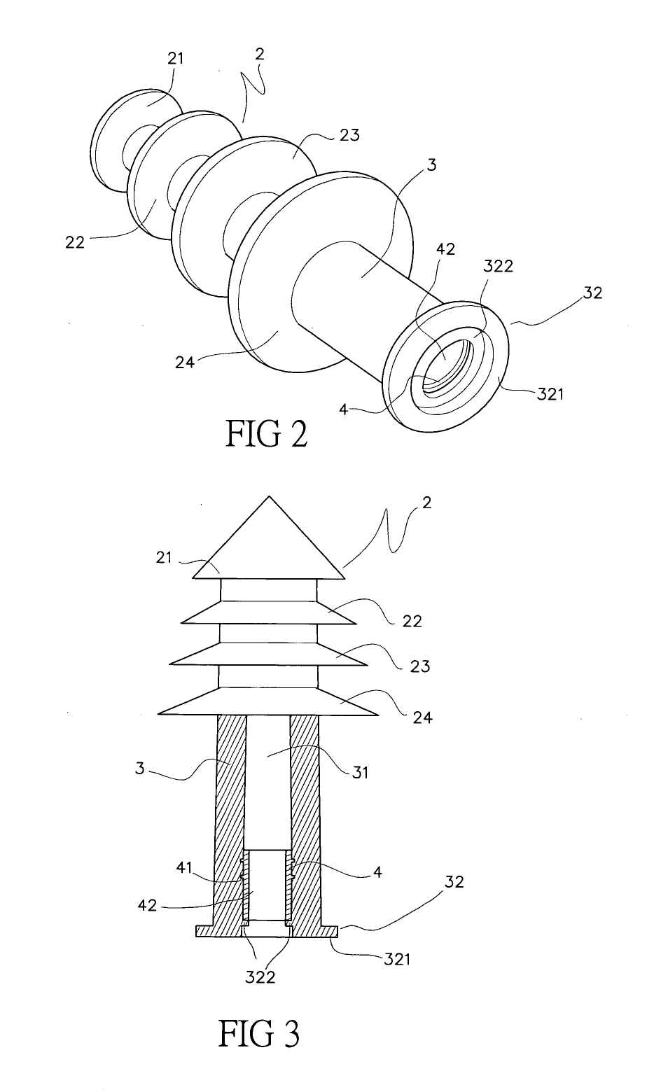

Please refer to FIGS. 2 and 3 that are assembled perspective and longitudinal sectional views, respectively, of an earplug according to a preferred embodiment of the present invention. As shown, the earplug includes a plug portion 2 and a handle portion 3. The plug portion 2 includes a plurality of axially sequentially arranged domed flanges 21, 22, 23 and 24.

The plug portion 2 extends rearward to a front end of the handle portion 3, and the handle portion 3 extends rearward by a predetermined length to internally define an axially extended hollow bore 31. An end cap 32 is externally formed around a rear end of the handle portion 3 to radially extend outward by a predetermined length to form a first flange 321. A second flange 322 is internally formed around the hollow bore 31 near the rear end of the handle portion 3 to radially extend inward by a predetermined length.

In the process of manufacturing the earplug, a metal-made hollow round tube 4 having two open ends is fixedly embedded in the hollow bore 31 of the handle portion 3 near the rear end thereof. With the second flange 322, the metal round tube 4 is prevented from moving out of the rear end of the hollow bore 31. As can be seen from FIG. 3, the metal round tube 4 is provided on an outer surface thereof with a plurality of circumferentially extended ribs 41 to tightly contact with an inner wall surface of the hollow bore 31, and has a rear end abutted on and accordingly, stopped by the second flange 322 from falling off the hollow bore 31. The first flange 321 of the end cap 32 allows a user to easily hold the earplug for conveniently plugging or extracting the earplug in or from the user's ear canal.

The metal-made hollow round tube 4 internally defines an axially extended inner bore 42 between the two open ends. When the metal round tube 4 is embedded and held in the hollow bore 31 near the end cap 32 at the rear end of the handle portion 3, the inner bore 42 of the metal round tube 4 allows the whole hollow bore 31 of the handle portion 3 to remain communicating with an environment outside the end cap 32.

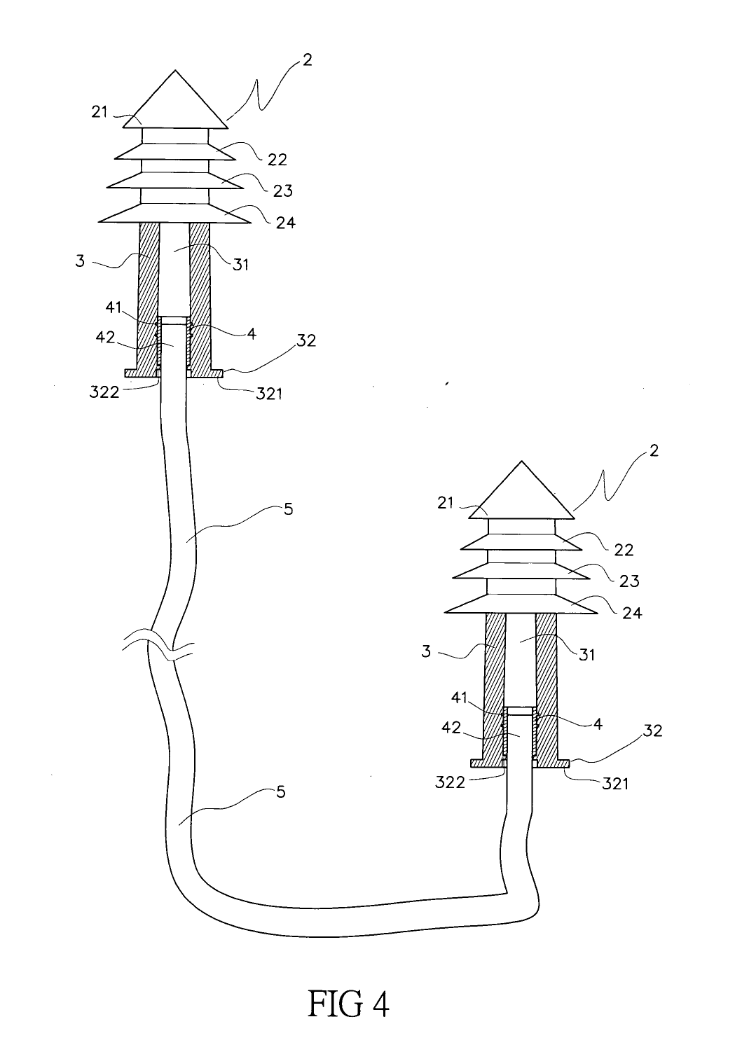

Please refer to FIG. 4. An extended cord 5 can be connected to two earplugs of the present invention by separately inserting and tight-fitting two opposite ends of the extended cord 5 in the inner bores 42 of the two earplugs, so that a user can wear the earplugs in a more convenient manner.

What is claimed is:

1. An earplug with embedded metal member, comprising a plug portion, a handle portion, and a metal-made hollow round tube; the plug portion including a plurality of axially sequentially arranged domed flanges and rearward extending to a front end of the handle portion; the handle portion extending rearward by a predetermined length to internally define an axially extended hollow bore with an end cap externally formed around a rear end of the handle portion; and the metal-made hollow round tube having two open ends and being fixedly embedded in the hollow bore of the handle portion near the end cap in the process of manufacturing the earplug.

2. The earplug as claimed in claim 1, wherein the metal-made hollow round tube is provided on an outer surface with a plurality of circumferentially extended ribs and internally defines an axially extended inner bore between the two open ends.

3. The earplug as claimed in claim 1, wherein the end cap of the handle portion radially extends outward by a predetermined length to form a first flange; and the hollow bore of the handle portion is internally provided near the rear end of the handle portion with a second flange that radially extends inward by a predetermined length.
